(19) United States
(12) Patent Application Publication
(10) Pub. No.: US 2025/0387220 A1
(43) Pub. Date: Dec. 25, 2025

(54) MULTIFOCAL INTRAOCULAR LENS

(71) Applicant: Vikrant BHALE, Aurangabad (IN)

(72) Inventor: Vikrant BHALE, Aurangabad (IN)

(21) Appl. No.: 18/879,198

(22) PCT Filed: Jun. 27, 2023
(86) PCT No.: PCT/IN2023/050625

§ 371 Date: Dec. 26, 2024

(30) Foreign Application Priority Data
Jun. 27, 2022 (IN) 202221028951

(51) Int. Cl. A61F 2/16

(52) U.S. Cl. CPC A61F 2/1618; A61F 2002/1681; A61F 2250/0053

(57) ABSTRACT

The multifocal intraocular lens 100 includes a first arm 105, a second arm 110 and an optic 115. The lens 100 is positioned on the pupil 120 instead of the cataractous natural lens of the eye. The optic 115 of the lens 100 has three different zones, each zone is defined with different degrees as per the user's requirement. The first zone 125 provide distance vision, the second zone 130 provides intermediate vision and the third zone 135 provides near vision. The zones defined on the optic 115 are adaptable to contraction and expansion of the pupil 120 and provide clear vision in both said states of the pupil 120.

### FIELD OF THE INVENTION

The present invention relates to intraocular lens and more particularly to intraocular lens for cataract surgeries with multiple focal lengths.

### BACKGROUND OF THE INVENTION

An intraocular lens (IOL) is a tiny, artificial lens for the human eye. It replaces the eye's natural lens that is removed during cataract surgery. The lens bends light rays that enter the eye, helping you to see. Presbyopia is a part of the normal aging process resulting in the hardening of the eye's natural lens and loss of function. This makes reading and focusing on near objects difficult without spectacles or contact lenses. Myopia, or short sightedness, is a result of the image focusing in front of the retina rather than on its surface, which causes poor distance vision, requiring specialist spectacles or contact lenses for correction. Hypermetropia, or long sightedness, results in the image focusing behind the retina rather than directly on its surface, which causes poor near vision, requiring specialist spectacles or contact lenses for correction.

US Patent Application US20070258143A1 to Valdemar Portney discloses a multifocal ophthalmic lens with an anterior and a posterior surface, a refractive zone and an aspherical base surface that produces multifocal powers. The multifocal lens with the refractive zone helps with near, intermediate and distant vision.

US Patent Application US2009088840A1 to Novartis AG discloses multifocal intraocular lens. The lens has a central refractive region that provides a refractive focusing power along with a diffractive region for providing two diffractive focusing powers. The invention particularly focuses on the focusing properties of the lens for far-focus ability, especially for small pupil sizes.

US Patent Application US20090292354A1 to STAAR SURGICAL CO discloses an optimized aspheric lens for patients having a curved retina. The invention discloses that a light entering the optimized aspheric lens on the axis at an angle to the optical axis is focused properly by the lens. This leads to reduction in aberrations and produces a much smaller spot size of light on the retina. The invention claims a method for optimizing parameters of intraocular lens adapted to be implanted into an eye of a patient.

Prior art suggests drawbacks in the side effects associated with diffractive structures such as glares and halos with loss of transmission due to diffractive lens. Prior art also suggests that loss of light transmission to IOL due to diffractive lens.

There is a need of a single intraocular lens to address Presbyopia, Myopia and Hypermetropia. There is a further need of a lens that passes adequate light for near, intermediate and distance vision of pupil size.

### SUMMARY OF THE INVENTION

A multifocal intraocular lens with multiple focal lengths having a first arm and a second arm for supporting the lens. An anterior side of the optic includes a plurality of zones for imparting clarity for distance, intermediate and near vision. A first zone is defined on the topmost portion of the optic, a second zone is defined on middle portion and lower middle portion of the optic and a third zone is defined on lowermost portion of the optic. The anterior side of the optic is circular in shape and has diameter in the range of 6 mm to 7.5 mm. The first zone of the optic ranges between a region defined by 180°-210°. The second zone of the optic ranges between a region defined by 20°-60°. The third zone of the optic ranges between a region defined by 90°-140°. The first, the second and the third zone have a transition zone ranging between 3.75°-5°. The first zone has diopter that ranges between 0.0 D to +40.0 D (A). The second zone has diopter that ranges between A+1.5-2.0 D. The diopter range for the third zone ranges between A+3.0-3.5 D.

### BRIEF DESCRIPTION OF DRAWINGS

The objectives and advantages of the present invention will become apparent from the following description read in accordance with the accompanying drawings wherein:

FIG. 1 shows a first zone in a multifocal intraocular lens in accordance with the present invention;

FIG. 2 shows a second zone in the multifocal intraocular lens of FIG. 1; and

FIG. 3 shows a third zone in the multifocal intraocular lens of FIG. 1.

### DETAILED DESCRIPTION OF THE INVENTION

The invention described herein is explained using specific exemplary details for better understanding. However, the invention disclosed can be worked on by a person skilled in the art without the use of these specific details.

References in the specification to “one embodiment” or “an embodiment” means that particular feature, structure, characteristic, or function described in connection with the embodiment is included in at least one embodiment of the invention. The appearances of the phrase “in one embodiment” in various places in the specification are not necessarily all referring to the same embodiment.

References in the specification to “preferred embodiment” means that a particular feature, structure, characteristic, or function described in detail thereby omitting known constructions and functions for clear description of the present invention.

The foregoing description of specific embodiments of the present invention has been presented for purposes of illustration and description. They are not intended to be exhaustive or to limit the present invention to the precise forms disclosed, and obviously many modifications and variations are possible in light of the above teaching.

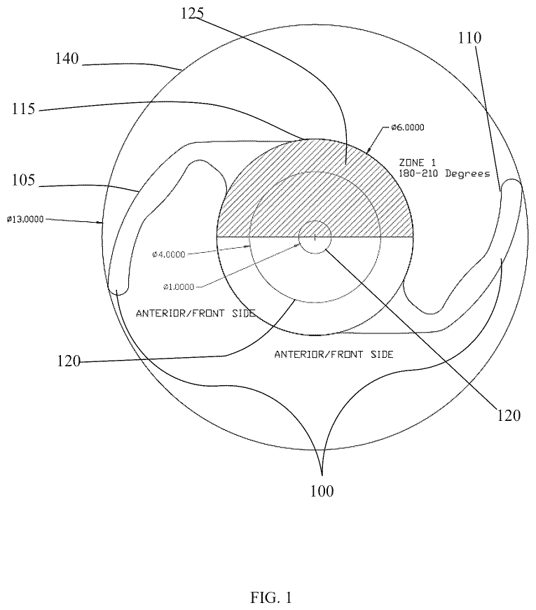

Referring to FIGS. 1, 2 and 3 an multifocal intraocular lens 100 in accordance with the present invention is described. The multifocal intraocular lens 100 is positioned inside the capsular bag 140. The multifocal lens replaces the cataractous natural lens in the capsular bag 140 to facilitate clear vision. The multifocal intraocular lens 100 (hereinafter referred as “the lens”) includes two opposed pair of arms for supporting the lens 100, i.e. a first arm 105 and a second arm 110. The lens 100 is implanted in the human eye by using methods in the prior art by expert eye surgeons. The lens 100 includes an optic 115 that is positioned behind a pupil 120. The first arm 105 and the second arm 110 is connected with the optic 115.

In accordance with the present invention, the anterior side of the optic 115 of the lens 100 includes three zones that are distinguished as per diopter of the respective zones. Accordingly, the zones are preferably engraved as a first zone 125, a second zone 130 and a third zone 135. It is to be noted that, each of the zones have a predefined range of degree for providing either near, distant or intermediate vision. The degrees of each zone are adaptable as per the user's requirement.

It is noted that, the pupil 120 has two states such as a dilated state and a contracted state. In accordance with the present invention, the lens 100 adapts to the dilated and contracted size of pupil 120 as per the situation. For example, the pupil 120 dilates in dark light environment and the pupil 120 contracts in bright light environment. In accordance with the present invention, the lens 100 advantageously focuses on distance, intermediate and near vision irrespective of the size of the pupil 120. This is because the radius of the curvature of the optic 115 is adapted in such a way that the distance vision is assigned with largest radius, the intermediate vision has radius ranged in between the distance and near vision and the near vision has the smallest radius. In other words, the lens 100 has multiple powers of vision at same point of time.

The first zone 125, the second zone 130 and the third zone 135 advantageously impart clear vision in both said states of the pupil 120. A person skilled in the art will appreciate that a contracted pupil 120 is preferably of 1.00 mm diameter whereas a dilated pupil 120 is preferably of 4.00 mm in diameter.

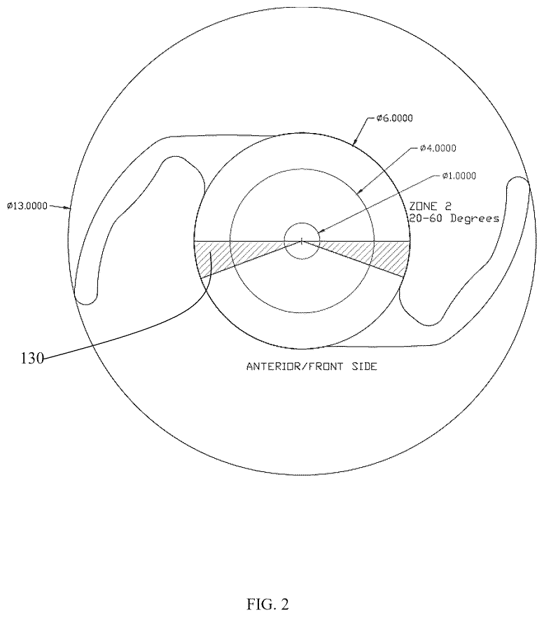

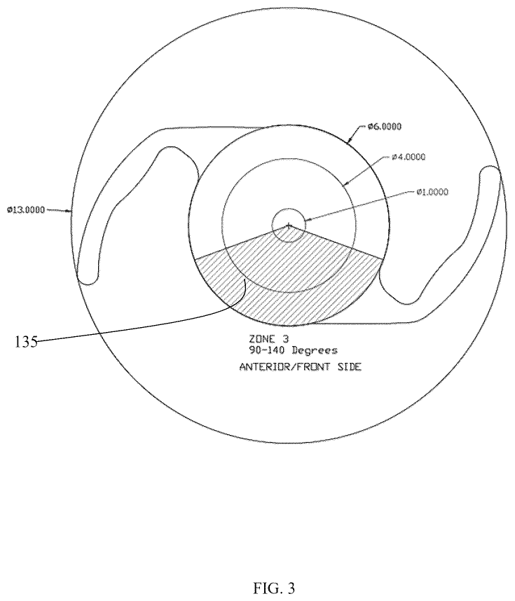

In this preferred embodiment, the anterior side of the optic 115 is circular in shape and preferably has diameter in the range of 6 mm to 7.5 mm. However, the size and shape of the optic 115 varies in other embodiments of the present invention according to the size of capsular bag of an individual. In this preferred embodiment, the first zone 125 (Refer FIG. 1) is defined on the topmost portion of optic 115. The second zone 130 (Refer FIG. 2) is defined in approximately middle portion and lower middle portion of the optic 115. Similarly, the third zone 135 (Refer FIG. 3) is defined on the lowermost portion of the optic 115. The zones 125, 130 and 135 are defined by manufacturing methods such as CNC and Precision Machining. It is noted however that, in other embodiments of the present invention, the positioning and defining of the zones may vary as per the requirement.

The optic 115 ranges up to 360° and the optic 115 includes zones 125-135 such that the sum of spread of all the zones 125-135 equals to 360°. In this one embodiment, the first zone 125 of the optic 115 ranges between a region defined by 180°-210°. The first zone 125 advantageously imparts clarity for distance vision through the pupil 120. The second zone 130 of the optic 115 ranges between a region defined by 20°-60°. The second zone 130 advantageously imparts clarity for intermediate vision through the pupil 120.

The second zone 130 is symmetrical on both sides of the optic 115. The second zone 130 is advantageously positionable on either side of the third zone 135 so as to transition from the first zone 125. The third zone 135 of the optic 115 ranges between a region defined by 90°-140°. The third zone 135 advantageously imparts clarity for near vision through the pupil 120. Each of the first 125, second 130 and third zone 135 have a transition zone ranging between 3.75°-5°. In accordance with the present invention, the transition zone is the blend radius when the radius changes from the first zone 125 to the second zone 130 and the second zone 130 to the third zone 135. The blend radius provides the smooth transition in the range between 3.75° to 5° to avoid sudden demarcation of the zones 125, 130 and 135 that advantageously minimizes glares, haloes or any such irregularities.

In accordance with the present invention, the lens 100 includes a plurality of zones in the optic combinations of the first 125, second 130 and third zone 135 in optic 115. It is well known in the prior art that, the diopter is a unit of refractive power, which is equal to the reciprocal of the focal length in meters of a given lens. Accordingly, the diopter range for first zone 125 ranges between 0.0 D to +40.0 D (A). The diopter range for second zone 130 ranges between A+1.5 to +2.0 D. The diopter range for third zone 135 ranges between A+3.0 to +3.5 D. The first zone 125 has largest radius, the second zone 130 has intermediate radius and the third zone 135 has smallest radius.

The multifocal intraocular lens 100, transmits adequate light for near, intermediate and distance vision to the pupil. The multifocal intraocular lens 100, is refractive in nature that provides better contrast, clearer distance, intermediate and near vision. The multifocal intraocular lens 100 advantageously, has minimal transition zones leading to less loss of light relative to conventional diffractive bifocal and trifocal IOL's. In the conventional diffractive bifocal and the trifocal IOL's the different steps and rings i.e., up to 15 rings are involved that leads to the loss of light reaching to retina. Whereas, in the present invention, the number of transition zones in the multifocal lens 100 are less. The transition zones advantageously reduce loss of light reaching to the retina. The multifocal intraocular lens 100 advantageously, has consistent depth of focus curves indicating extended depth of focus between intermediate and near vision.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the scope of the present invention.

I claim:

1. A multifocal intraocular lens 100 with multiple focal lengths having a first arm 105 and a second arm 110 for supporting the lens 100 characterized in that said lens 100 comprising:
an anterior side of the optic 115 including a plurality of zones for imparting clarity for distance, intermediate and near vision, a first zone 125 defined on the topmost portion of the optic 115;
a second zone 130 defined on middle portion and lower middle portion of the optic 115; and a third zone 135 defined on lowermost portion of the optic 115; and
the anterior side of the optic 115 being circular in shape and having diameter in the range of 6 mm to 7.5 mm.

2. The multifocal intraocular lens 100 as claimed in claim 1, wherein the first zone 125 of the optic 115 ranges between a region defined by 180°-210°.

3. The multifocal intraocular lens 100 as claimed in claim 1, wherein the second zone 130 of the optic 115 ranges between a region defined by 20°-60°.

4. The multifocal intraocular lens 100 as claimed in claim 1, wherein the third zone 135 of the optic 115 ranges between a region defined by 90°-140°.

5. The multifocal intraocular lens 100 as claimed in claim 1, wherein the first 125, the second 130 and the third zone 135 have a transition zone ranging between 3.75° to 5°.

6. The multifocal intraocular lens 100 as claimed in claim 1, wherein the first zone 125 including diopter that ranges between 0.0 D to +40.0 D (A).

7. The multifocal intraocular lens 100 as claimed in claim 1, wherein the second zone 130 including diopter that ranges between A+1.5 to +2.0 D.

8. The multifocal intraocular lens 100 as claimed in claim 1, wherein the diopter range for the third zone 135 ranges between A+3.0 to +3.5 D.
